(19) United States
(12) Patent Application Publication
(10) Pub. No.: US 2025/0387780 A1
(43) Pub. Date: Dec. 25, 2025

(54) METHOD FOR PREPARING SUPPORTED TRANSITION METAL CATALYST, SUPPORTED TRANSITION METAL CATALYST AND USE THEREOF IN CONDENSATION COUPLING SYNTHESIS OF HIGH-CARBON KETONE FROM ALPHA-H-CONTAINING KETONE AND ALCOHOL

(71) Applicant: Zhejiang Sainon Chemical Co., Ltd., Zhejiang (CN)

(72) Inventors: Jianhui LI, Zhejiang (CN); Shuqun XU, Zhejiang (CN); Weipeng LI, Zhejiang (CN); Lizhong ZHANG, Zhejiang (CN); Ji'an FAN, Zhejiang (CN)

(21) Appl. No.: 19/010,675

(22) Filed: Jan. 6, 2025

(30) Foreign Application Priority Data
Jun. 24, 2024 (CN) 202410817116.8
Aug. 16, 2024 (CN) 202411125706.0

(51) Int. Cl. B01J 23/755; B01J 23/80; B01J 23/84; B01J 37/04; B01J 37/08; B01J 37/18; C07C 45/45; C07C 45/71; C07C 49/04

(52) U.S. Cl. CPC B01J 23/755; B01J 23/80; B01J 23/84; B01J 37/04; B01J 37/088; B01J 37/18; C07C 45/45; C07C 45/71; C07C 49/04

(57) ABSTRACT

A method for preparing a supported transition metal catalyst, and the supported transition metal catalyst and use thereof in condensation coupling synthesis of a high-carbon ketone from an α-H-containing ketone and an alcohol are provided. Preparation process of the supported transition metal catalyst includes adding a porous catalyst carrier to a solution of a transition metal salts, followed by standing, drying, calcining, and reducing. The transition metal salt is at least one selected from the group consisting of transition metal nitrates, transition metal formates, transition metal oxalates, and transition metal acetates, and the transition metal is a non-noble metal selected from the group consisting of transition metal elements from Groups VIIB, VIII, IB and IIB of the periodic table of the chemical elements.

### CROSS REFERENCE TO RELATED APPLICATION

This patent application claims the benefit and priority of Chinese Patent Application No. 202410817116.8 filed with the China National Intellectual Property Administration on Jun. 24, 2024, and Chinese Patent Application No. 202411125706.0 filed with the China National Intellectual Property Administration on Aug. 16, 2024, these disclosures of which are incorporated by reference herein in their entirety as part of the present application.

### TECHNICAL FIELD

The present disclosure relates to the technical field of catalysis, and in particular to a method for preparing a supported transition metal catalyst and the supported transition metal catalyst and use thereof in condensation coupling synthesis of a high-carbon ketone from an alpha (α)-H-containing ketone and an alcohol.

### BACKGROUND

Ketones serve as excellent organic solvents and also as important organic chemical raw materials in the synthesis of perfumes, organic compounds, and pharmaceutical intermediates, and the like. Existing ketone synthesis methods in the prior art are limited, often involving cumbersome processes and producing numerous by-products. Traditional preparation methods include oxidative dehydrogenation of alcohols and aldol condensation followed by hydrogenation. These methods are costly and result in low product selectivity. For instance, U.S. Pat. No. 4,146,581 discloses a process for producing pentanone from acetaldehyde and acetone through condensation, followed by dehydration and hydrogenation in the presence of hydrogen, which yields complex product components with low target product selectivity and requires high-pressure hydrogen, demanding stringent reaction conditions.

The size of a ketone molecule influences its reactivity and the energy released during reaction. High-carbon ketones, compared to their small-molecule counterparts, possess a relatively larger molecular structure that facilitates more complex and diverse chemical reactions. It has been disclosed in the prior art that α-H-containing ketones react with other small-molecule carbonyl compounds such as ketones and aldehydes with reactive α-H groups, to form condensation-coupling products. These products can subsequently be dehydrated and hydrogenated to yield alkyl-grafted high-carbon ketones. Alcohols are also utilized as condensation coupling reagents in the preparation of high-carbon ketones through coupling with α-H-containing ketones. The high-carbon ketone reactions detailed in the prior art described above all involve four steps: hydrogen borrowing (dehydrogenation of alcohol to produce a small-molecule carbonyl compound with an active α-H), aldol condensation (using the α-H-containing ketone as a substrate), dehydration, and hydrogenation. These steps place significant demands on catalyst design. Catalyst systems reported in the prior art encompass supported noble metal catalysts, homogeneous catalysts, and complex oxide catalysts. For instance, CN 106732555 A describes the use of a Pd/C catalyst for the α-alkylation of a ketone with an alcohol, which requires the solvent of 1,4-dioxane when catalyzing the reaction between acetophenone with n-butanol. CN 111889105 A discloses a bifunctional catalyst for the alkylation of methanol and butanone to produce 3-pentanone, comprising 2-30% nickel oxide, 40-90% magnesium oxide, 10-30% aluminum oxide, and 0-20% zinc oxide, with a reaction temperature of 220-350° C. and a methanol/butanone molar ratio of 5-15:1. CN 110423190 A and CN 106905125 A report iron and cobalt complexes, respectively, for catalyzing α-alkylation of ketones. The catalysts are synthesized by reacting 4′-dimethylaminophenyl-2,2′: 6′,2″-terpyridine with anhydrous ferrous chloride and 2,2;6,2″-terpyridine with cobalt chloride, respectively. It is evident that these publicly available technologies face issues such as high catalyst costs, complex and severe reaction conditions, and challenges in product separation and catalyst recycling.

### SUMMARY

To address the aforementioned issues, the present disclosure provides a method for preparing a supported transition metal catalysts, and the supported transition metal catalyst and use thereof in condensation coupling synthesis of a high-carbon ketone from an α-H-containing ketone and an alcohol.

In a first aspect, the present disclosure provides a method for preparing a supported transition metal catalyst, the method including the following steps:
S1: dissolving at least one transition metal salt in water to form a solution, the transition metal salt being at least one selected from the group consisting of transition metal nitrates, transition metal formates, transition metal oxalates, and transition metal acetates;
S2: adding a porous catalyst carrier to the solution obtained in step S1 and stirring at atmospheric temperature to be uniform to obtain a mixed solution;
S3: placing the mixed solution obtained in step S2 in an oven and drying at a temperature of 80-110° C. to a constant weight to obtain a dry mass;
S4: heating the dry mass obtained in step S3 to a calcining temperature of 250-390° C. in a calcining device and calcining for 5-8 hours to obtain a calcined product; and
S5: heating the calcined product obtained in step S4 to a reduction temperature of 250-450° C. in a reaction device and reducing in a reducing atmosphere for 2-8 hours to obtain the supported transition metal catalyst, where a molar ratio of the transition metal salt (n1), the porous catalyst carrier (n2), and the water as a solvent (n3), i.e., n1:n2:n3, is in a range of not less than 1:5:50 to not more than 1:20:100.

In certain embodiments, the molar ratio of the transition metal salt (n1), the porous catalyst carrier (n2), and the water as a solvent (n3), i.e., n1:n2:n3, is in a range of not less than 1:8:60 to not more than 1:12:80.

In certain embodiments, in step S4, a heating rate in the calcining device is 2-10° C. per minute, and the calcining temperature is 300-350° C.

In certain embodiments, in step S5, a heating rate in the reaction device is 2-10° C. per minute, and the reduction temperature is 300-350° C.

In certain embodiments, transition metals in the transition metal nitrates and the transition metal acetates are each at least one selected from the group consisting of Mn, Ni, Co, Fe, Zn, and Cu. In certain embodiments, the transition metal is at least one selected from the group consisting of Ni, Co, and Cu.

In certain embodiments, the transition metal salt is at least two selected from the group consisting of the transition metal nitrates, the transition metal formates, the transition metal oxalates, and the transition metal acetates.

In certain embodiments, the porous catalyst carrier is at least one material selected from the group consisting of porous materials; preferably at least one selected from the group consisting of porous carbon materials, carbon nanotubes, alkaline earth oxides, aluminum oxide, silicon oxide, aluminum silicon oxide, and diatomaceous earth; and more preferably, the porous catalyst is at least one selected from the group consisting of activated carbon, and magnesium oxide.

In certain embodiments, the reducing atmosphere is a mixture of H₂ and N₂ gases; and a volume fraction of H₂ in the mixture of H₂ and N₂ gases is 5% to 50%.

In a second aspect, the present disclosure provides a supported transition metal catalyst prepared by the method described above.

In certain embodiments, the supported transition metal catalyst includes any one selected from the group consisting of Ni₅-Fe₁/AC, Ni₂-Fe₁/AC, Ni₅-Co₁/AC, Ni₅-Cu₁/AC, Co₅—Zn/AC, Mn₅—Cu₁/AC, Ni₅/AC, Co₂/AC, Ni₅-Fe₁/CNT, and Ni₅-Fe₁/MgO.

In a third aspect, the present disclosure further provides a method for catalytic synthesis of a high-carbon ketone, which includes: in a closed reaction device, using the supported transition metal catalyst prepared by the method described above as a reaction catalyst, and an α-H-containing ketone and an alcohol as reactive substrates; and conducting condensation coupling reaction at a starting pressure of atmospheric pressure and a reaction temperature of 120-250° C. to obtain the high-carbon ketone.

In certain embodiments, the alcohol is at least one selected from the group consisting of aliphatic alcohols, aromatic alcohols, alicyclic alcohols, and alcohols containing an additional heteroatom substituent group; and preferably, the alcohol is at least one selected from the group consisting of ethanol, n-propanol, isopropanol, ethylene glycol, phenylethyl alcohol, cyclohexanol, and ethanolamine.

In certain embodiments, the α-H-containing ketone is at least one selected from the group consisting of aliphatic ketones, aromatic ketones, alicyclic ketones, and ketones containing an additional heteroatom substituent group; and preferably, the α-H-containing ketone is at least one selected from the group consisting of acetone, butanone, pentanone, acetophenone, cyclohexanone, and 1-amino-2-propanone.

In certain embodiments, a molar ratio of the α-H-containing ketone to the alcohol is in a range of 1:2 to 2:1, and a feeding ratio of the α-H-containing ketone to the supported transition metal catalyst is 0.2-0.3 grams of the supported transition metal catalyst used per 1 mole of the α-H-containing ketone.

In certain embodiments, the condensation coupling reaction may be performed using a kettle reactor, a fixed bed process, or a fluidized bed process, and the like. In certain embodiments, the condensation coupling reaction is conducted as a continuous process with simultaneous feeding and discharging; and more preferably the condensation coupling reaction is conducted at a temperature of 160-210° C. for 30-300 minutes.

Transition metal catalysts provided by the present disclosure could efficiently catalyze condensation coupling reaction between a ketone and a small-molecule alcohol, yielding a target high-carbon ketone with high selectivity. Additionally, metal used for the catalyst is a non-noble metal, and raw materials for the catalyst are readily available, thus significantly reducing the catalysts's cost. Utilizing the catalyst of the present disclosure for condensation reaction between a ketone and alcohol, a reaction process is easy to achieve without the need for an additional solvent and high-pressure hydrogen, thus simplifying the preparation process. Furthermore, the catalyst allows for a broad range of the alcohols to be applicable to the condensation coupling reaction with high conversion rates, achieving up to 80% or more conversion rates for both alcohol and ketone, and up to 90% or more selectivity towards the target high-carbon ketone. The catalyst also exhibits stable performance over an extended period, indicating promising prospects for industrial application.

### DETAILED DESCRIPTION OF THE EMBODIMENTS

To facilitate a comprehensive understanding of the technical solution of the present disclosed, detailed descriptions of certain embodiments of the present disclosed are provided below with reference to the drawings.

It should be clear that the described embodiments are just some embodiments of the present disclosure, not all of them. Based on the embodiments in the present disclosure, all the other embodiments that would have been obtained by those of ordinary skill in the art without any inventive effort shall fall within the scope of the present disclosure.

The terminology employed in the embodiments of the present disclosure is for the purpose of describing particular embodiments and is not intended to limit the scope of the present disclosure. Unless clearly indicated otherwise within the context, the singular forms “a,” “an,” and “the” used in the embodiments of the present disclosure and the appended claims include the plural forms as well.

It should be understood that the term “and/or” as used herein is merely a description of the association relationship between related objects, indicating that there may be three types of relationship, such as A and/or B, which may represent three situations: A alone, A and B together, or B alone. In addition, the character “/” as used herein generally indicates that the related objects before and after are in an “or” relationship.

In the description of this specification, it is to be understood that the words “substantially”, “approximately”, “about”, “around”, “roughly”, “generally”, and the like used in the claims and embodiments of the present disclosure, are intended to encompass values that fall within a reasonable range of process operation or tolerance, rather than implying exact numerical values.

The present disclosure is further described below in combination with specific embodiments, but is not limited thereto.

The method for preparing a supported transition metal catalyst, including the following steps:
S1: dissolving at least one transition metal salt in water to form a solution, where the transition metal salt is at least one selected from the group consisting of transition metal nitrates, transition metal formates, transition metal oxalates, and transition metal acetates;
S2: adding a porous catalyst carrier to the solution obtained in step S1, and stirring at atmospheric temperature to obtain a uniform mixed solution;
S3: placing the mixed solution obtained in step S2 in an oven and drying at a temperature of 80-110° C. to a constant weight to obtain a dry mass;
S4: heating the dry mass obtained in step S3 to a calcining temperature of 250-390° C. in a calcining device and calcining for 5-8 hours to obtain a calcined product; and
S5: heating the calcined product obtained in step S4 to a reduction temperature of 250-450° C. in a reaction device and reducing in a H₂/N₂ mixed gas stream for 2-8 hours to obtain the supported transition metal catalyst.

In certain embodiments, a molar ratio of the transition metal salt (n1), the porous catalyst carrier (n2), and the water as a solvent (n1), i.e., n1:n2:n3 is in a range of not less than 1:5:50 to not more than 1:20:100; and preferably, not less than 1:8:60 to not more than 1:12:80.

In certain embodiments, during step S4, the heating rate in the calcining device is between 2-10° C. per minute, and the calcining temperature is maintained between 300-350° C.

In certain embodiments, in step S5, a heating rate in the reaction device is 2-10° C. per minute, and the reduction temperature is 300-350° C.

In certain embodiments, a transition metal element in the transition metal salt is selected from the group consisting of the transition metal elements from Groups VIIB, VIII, IB and IIB of the periodic table of the chemical elements, and the transition metal is a non-noble metal. In certain embodiments, the transition metal is at least one selected from the group consisting of Mn, Ni, Co, Fe, Zn, and Cu. In other embodiments, the transition metal is at least one selected from the group consisting of Ni, Co, and Cu.

In certain embodiments, the transition metal salt is at least two selected from the group consisting of the transition metal nitrates, the transition metal formates, the transition metal oxalates, and the transition metal acetates.

The transition metal used for the catalyst of the present disclosure is the non-noble metal, and raw materials for the catalyst are readily available, thereby significantly reducing the cost of the catalyst.

In certain embodiments, the porous catalyst carrier is at least one selected from the group consisting of porous carbon materials, carbon nanotubes, alkaline earth oxides, aluminum oxide, silicon oxide, aluminum silicon oxide, and diatomaceous earth; and preferably, the porous catalyst carrier is at least one selected from the group consisting of porous carbon and magnesium oxide.

The porous catalyst carrier has small connecting pores between its constituent pores, which confine the catalyst within the pores after loading and reduce the likelihood of agglomerate, thereby significantly enhancing the catalyst's lifespan.

### EXEMPLARILY, REFERENCE TO EXAMPLES 1-10

### Example 1

Nickel nitrate hexahydrate and iron nitrate nonahydrate were dissolved in water, activated carbon was added thereto to form a mixture. The mixture was stirred at atmospheric temperature for 7-10 hours, then placed in an oven at 100° C. and dried to a constant weight. A molar ratio of the nickel nitrate hexahydrate, the iron nitrate nonahydrate, the activated carbon, and water was 5:1:50:320. After drying to a constant weight, a resulting product was placed in a muffle furnace and calcined under an air atmosphere at 300° C. for 6 hours with a heating rate of 5° C. per minute, and subsequently reduced in a 5% H₂/N₂ mixed gas stream at 400° C. for 8 hours (with a heating rate of 5° C. per minute) to yield a catalyst, labeled as Ni₅-Fe₁/AC.

### Examples 2-6

The same method as in catalyst preparation of example 1 was used, except that transition metal nitrates and proportions thereof, and reaction conditions. The calcining temperature were set to 300° C., 250° C., 350° C., 330° C., and 300° C., respectively; the heating rate were set to 5° C. per minute, 7° C. per minute, 9° C. per minute, 3° C. per minute, and 5° C. per minute, respectively; and the reduction temperature were set to 400° C., 300° C., 250° C., 450° C., and 400° C., respectively.

Resulting catalysts were labeled as Ni₂-Fe₁/AC, Ni₅-Co₁/AC, Ni₅-Cu₁/AC, Co₅—Zn₁/AC, and Mn₅—Cu₁/AC, respectively. Specific feeding ratios are provided in Table 1 below.

### Examples 7-8

The same method and reaction conditions as in catalyst preparation of example 1 were used, except that catalyst carriers were changed to carbon nanotubes (CNT) and magnesium oxide (MgO), respectively. Resulting catalysts were labeled as Ni₅-Fe₁/CNT and Ni₅-Fe₁/MgO, respectively.

### Examples 9-10

The same method and reaction conditions as in catalyst preparation of example 1 were used, except that a single transition metal nitrate was used as a substrate. Resulting catalysts were labeled as Ni₅/AC and Co₂/AC, respectively.

TABLE 1
Table of substrate feeding for preparation
of transition metal catalyst

| | Transition metal nitrate | | | |
|---|---|---|---|---|
| | | Molar | Catalyst | Resulting |
| Example | Type | ratio | carrier | catalyst |
| Example | Nickel nitrate:iron | 5:1 | Activated | Ni₅—Fe₁ |
| 1 | nitrate | | carbon (AC) | |
| Example | Nickel nitrate:iron | 2:1 | Activated | Ni₂—Fe₁ |
| 2 | nitrate | | carbon (AC) | |
| Example | Nickel acetate:cobalt | 5:1 | Activated | Ni₅—Co₁ |
| 3 | nitrate | | carbon (AC) | |
| Example | Nickel nitrate:copper | 5:1 | Activated | Ni₅—Cu₁ |
| 4 | acetate | | carbon (AC) | |
| Example | Cobalt nitrate:zinc | 5:1 | Activated | Co₅—Zn₁ |
| 5 | nitrate | | carbon (AC) | |
| Example | Manganese nitrate:copper | 5:1 | Activated | Mn₅—Cu₁ |
| 6 | nitrate | | carbon (AC) | |
| Example | Nickel nitrate:iron | 5:1 | Carbon | Ni₅—Fe₁/ |
| 7 | nitrate | | nanotubes | CNT |
| | | | (CNT) | |
| Example | Nickel nitrate:iron | 5:1 | Magnesium | Ni₅—Fe₁/ |
| 8 | nitrate | | oxide (MgO) | MgO |
| Example | Nickel nitrate | 5 | Activated | Ni₅/AC |
| 9 | | | carbon (AC) | |
| Example | Cobalt nitrate | 2 | Activated | Co₂/AC |
| 10 | | | carbon (AC) | |

Additionally, the present disclosure provides a method for catalytic synthesis of a high-carbon ketone, which includes: in a closed reaction device, using the supported transition metal catalyst described above as a reaction catalyst, and an α-H-containing ketone and an alcohol as reactive substrates; and conducting condensation coupling reaction at a starting pressure of atmospheric pressure and a reaction temperature of 120-250° C. to obtain the high-carbon ketone.

In certain embodiments, the alcohol may be at least one selected from the group consisting of aliphatic alcohols, aromatic alcohols, alicyclic alcohols, and alcohols containing an additional heteroatom substituent group; and preferably, the alcohol is at least one selected from the group consisting of ethanol, n-propanol, isopropanol, ethylene glycol, phenylethyl alcohol, cyclohexanol, and ethanolamine.

In certain embodiments, the α-H-containing ketone is at least one selected from the group consisting of aliphatic ketones, aromatic ketones, alicyclic ketones, and ketones containing an additional heteroatom substituent group; and preferably, the α-H-containing ketone is at least one selected from the group consisting of acetone, butanone, pentanone, acetophenone, cyclohexanone, and 1-amino-2-propanone.

The condensation coupling reaction may be conducted by using a kettle reactor, a fixed-bed process, or a fluidized bed process, and the like. In certain embodiments, the reaction is conducted as a continuous process with simultaneous feeding and discharging. Furthermore, the process operation steps could be simplified to facilitate mass industrial production.

In certain embodiments, a molar ratio of the α-H-containing ketone to the alcohol is in a range of 1:2 to 2:1, and a feeding ratio of the α-H-containing ketone to the supported transition metal catalyst is 0.2-0.3 grams of the supported transition metal catalyst added per 1 mol of the α-H-containing ketone.

In certain embodiments, the condensation coupling reaction is conducted as a continuous process with simultaneous feeding and discharging; and preferably, the condensation coupling reaction is conducted at a temperature of 160-210° C. for 30-300 minutes.

### Exemplarily, Reference can be Made to the Following Examples 11-31 of Condensation Coupling Reaction

### Example 11 of the Condensation Coupling Reaction

In a 100 ml autoclave, 0.05 g of a catalyst Ni₅-Fe₁/AC, 20 ml of ethanol, and 20 ml of acetone were sequentially added and subjected to reaction at 175° C. for 1.7 hours, during which a rotation speed of a stirring device was maintained at 500 revolutions per minute (rpm). Post-reaction gas chromatography analysis reveals that a conversion rate of ethanol is 92%, a conversion rate of acetone is 87%, and a selectivity towards 2-pentanone is 83%.

### Examples 12-31 of Condensation Coupling Reaction

Methods similar to that of the reaction of example 11 were used, except that reaction conditions and substrates were changed. The composition of products after the reaction was analyzed. The reaction conditions and catalytic performance results for the condensation coupling reaction in Examples 12-31 are detailed in Table 2 below.

TABLE 2
Reaction conditions of condensation coupling reaction of examples and catalytic performance results

| | | | | | | | | % |
|---|---|---|---|---|---|---|---|---|
| | | | | | | | | Selectivity |
| | | | | | | % | % | towards |
| | | | | | | Conversion | Conversion | corresponding |
| | | | | | | rate of | rate of | high- |
| | | | Time | Substrate | Substrate | substrate | substrate | carbon |
| Example | Catalyst | Temperature | h | of ketone | of alcohol | of ketone | of alcohol | ketone |
| 11 | Ni₅—Fe₁/AC | 175 | 1.7 | Acetone | Ethanol | 87 | 92 | 93 |
| 12 | Ni₅—Fe₁/AC | 175 | 1.0 | Acetone | Ethanol | 88 | 81 | 91 |
| 13 | Ni₅—Fe₁/AC | 175 | 1 | Acetone | Ethanol | 82 | 87 | 94 |
| 14 | Ni₅—Fe₁/AC | 175 | 0.5 | Acetone | Ethanol | 87 | 89 | 90 |
| 15 | Ni₅—Fe₁/AC | 190 | 1 | Acetone | Ethanol | 93 | 88 | 92 |
| 16 | Ni₅—Fe₁/AC | 175 | 1 | Acetone | n- | 80 | 85 | 90 |
| | | | | | Propanol | | | |
| 17 | Ni₅—Fe₁/AC | 175 | 1 | Acetone | Ethylene | 81 | 83 | 89 |
| | | | | | glycol | | | |
| 18 | Ni₅—Fe₁/AC | 175 | 1 | Acetone | Glycerol | 80 | 81 | 91 |
| 19 | Ni₅—Fe₁/AC | 175 | 1 | Butanone | Ethanolamine | 80 | 86 | 93 |
| 20 | Ni₅—Fe₁/AC | 175 | 1 | 2- | Ethanol | 76 | 85 | 92 |
| | | | | Pentanone | | | | |
| 21 | Ni₅—Fe₁/AC | 175 | 1 | Acetophenone | Ethanol | 82 | 86 | 90 |
| 22 | Ni₅—Fe₁/AC | 175 | 1 | Butanone | n- | 81 | 80 | 92 |
| | | | | | Propanol | | | |
| 23 | Ni₅—Fe₁/AC | 175 | 1 | Cyclohexanone | Ethanol | 85 | 83 | 93 |
| 24 | Ni₅—Fe₁/AC | 175 | 1 | Butanone | Isopropanol | 88 | 85 | 90 |
| 25 | Ni₅—Fe₁/AC | 175 | 1 | Acetone | Ethylene | 92 | 87 | 91 |
| | | | | | glycol | | | |
| 26 | Co₅—Zn₁/AC | 175 | 1 | Acetone | Phenylethyl | 77 | 83 | 88 |
| | | | | | alcohol | | | |
| 27 | Mn₅—Cu₁/AC | 175 | 1 | Acetone | Cyclohexanol | 82 | 87 | 90 |
| 28 | Ni₅—Fe₁/CNT | 175 | 1 | 1-Amino- | Ethanol | 85 | 90 | 91 |
| | | | | 2-propanone | | | | |
| 29 | Ni₅—Fe₁/MgO | 175 | 1 | Acetone | Ethanol | 72 | 77 | 81 |
| 30 | Ni₅/AC | 175 | 1 | Acetone | Ethanol | 61 | 70 | 87 |
| 31 | Co₂/AC | 175 | 1 | Acetone | Ethanol | 66 | 67 | 89 |

When the catalyst of the present disclosure is utilized to catalyze the condensation reaction between a ketone and an alcohol, the reaction process is easy to achieve without the need for an additional solvent and high-pressure hydrogen, thus simplifying the preparation process. Furthermore, the catalyst allows for a broad range of alcohols to be effectively used in the condensation coupling reaction with high conversion rates, achieving up to 80% or greater conversion rates for both alcohol and ketone, and up to 90% or greater towards the target high-carbon ketone. The catalyst also exhibits stable performance over an extended period, indicating favorable prospects for industrial application.

What is claimed is:

1. A method for preparing a supported transition metal catalyst, the method comprising:
S1: dissolving at least one transition metal salt in water to form a solution, wherein the transition metal salt is at least one selected from the group consisting of a transition metal nitrate, a transition metal formate, a transition metal oxalate, and a transition metal acetate;
S2: adding a porous catalyst carrier to the solution obtained in step S1 and stirring at atmospheric temperature to be uniform to obtain a mixed solution;
S3: placing the mixed solution obtained in step S2 in an oven and drying at a temperature of 80-110° C. to a constant weight to obtain a dry mass;
S4: heating the dry mass obtained in step S3 to a calcining temperature of 250-390° C. in a calcining device and calcining for 5-8 hours to obtain a calcined product; and
S5: heating the calcined product obtained in step S4 to a reduction temperature of 250-450° C. in a reaction device and reducing in a reducing atmosphere for 2-8 hours to obtain the supported transition metal catalyst,
wherein a molar ratio of the transition metal salt (n1), the porous catalyst carrier (n2), and the water as a solvent (n3) is in a range of not less than 1:5:50 to not more than 1:20:100.

2. The method of claim 1, wherein in step S4, a heating rate in the calcining device is 2-10° C. per minute, and the calcining temperature is 300-350° C.

3. The method of claim 1, wherein in step S5, a heating rate in the reaction device is 2-10° C. per minute, and the reduction temperature is 300-350° C.

4. The method of claim 1, wherein transition metals in the transition metal nitrate, the transition metal formate, the transition metal oxalate, and the transition metal acetate are each at least one selected from the group consisting of Mn, Ni, Co, Fe, Zn, and Cu.

5. The method of claim 1, wherein the transition metal salt is at least two selected from the group consisting of the transition metal nitrate, the transition metal formate, the transition metal oxalate, and the transition metal acetate.

6. The method of claim 1, wherein the porous catalyst carrier is at least one selected from the group consisting of a porous carbon material, a carbon nanotube, a alkaline earth oxide, a silicon oxide, an aluminum silicon oxide, and diatomaceous earth.

7. The method of claim 1, wherein the reducing atmosphere is a H₂/N₂ mixed gas stream; and a volume fraction of H₂ in the H₂/N₂ mixed gas stream is 5%-50%.

8. A supported transition metal catalyst prepared by the method of claim 1.

9. The supported transition metal catalyst of claim 8, wherein the supported transition metal catalyst is any one selected from the group consisting of Ni₅-Fe₁/AC, Ni₂-Fe₁/AC, Ni₅-Co₁/AC, Ni₅-Cu₁/AC, Co₅—Zn/AC, Mn₅—Cu₁/AC, Ni₅/AC, Co₂/AC, Ni₅-Fe₁/CNT, and Ni₅-Fe₁/MgO.

10. A method for catalytic synthesis of a high-carbon ketone, comprising:
in a closed reaction device, using the supported transition metal catalyst of claim 8 as a catalyst, and an α-H-containing ketone and an alcohol as reactive substrates; and conducting condensation coupling reaction at a starting pressure of atmospheric pressure and a reaction temperature of 120-250° C. to obtain the high-carbon ketone.

11. The method of claim 10, wherein the alcohol is at least one selected from the group consisting of an aliphatic alcohol, an aromatic alcohol, an alicyclic alcohol, and an alcohol containing additional heteroatom substituent group.

12. The method of claim 10, wherein the α-H-containing ketone is at least one selected from the group consisting of an aliphatic ketone, an aromatic ketone, an alicyclic ketone, and a ketone containing additional heteroatom substituent group.

13. The method of claim 10, wherein a molar ratio of the α-H-containing ketone to the alcohol is in a range of 1:2 to 2:1; and a feeding ratio of the α-H-containing ketone to the supported transition metal catalyst is 0.2-0.3 grams of the supported transition metal catalyst added per 1 mole of the α-H-containing ketone.

14. The method of claim 10, wherein the condensation coupling reaction may be conducted using a kettle reactor, a fixed bed process, or a fluidized bed process;
the condensation coupling reaction is conducted as a continuous process with simultaneous feeding and discharging; and
the condensation coupling reaction is conducted at a temperature of 160-210° C. for 30-300 minutes.

15. The supported transition metal catalyst of claim 8, wherein in step S4, a heating rate in the calcining device is 2-10° C. per minute, and the calcining temperature is 300-350° C.

16. The supported transition metal catalyst of claim 8, wherein in step S5, a heating rate in the reaction device is 2-10° C. per minute, and the reduction temperature is 300-350° C.

17. The supported transition metal catalyst of claim 8, wherein transition metals in the transition metal nitrate, the transition metal formate, the transition metal oxalates, and the transition metal acetate are each at least one selected from the group consisting of Mn, Ni, Co, Fe, Zn, and Cu.

18. The supported transition metal catalyst of claim 8, wherein the transition metal salt is at least two selected from the group consisting of the transition metal nitrate, the transition metal formate, the transition metal oxalate, and the transition metal acetate.

19. The supported transition metal catalyst of claim 8, wherein the porous catalyst carrier is at least one selected from the group consisting of a porous carbon material, a carbon nanotube, an alkaline earth oxide, a silicon oxide, an aluminum silicon oxide, and diatomaceous earth.

20. The method of claim 10, wherein the supported transition metal catalyst is any one selected from the group consisting of Ni₅-Fe₁/AC, Ni₂-Fe₁/AC, Ni₅-Co₁/AC, Ni₅-Cu₁/AC, Co₅—Zn/AC, Mn₅—Cu₁/AC, Ni₅/AC, Co₂/AC, Ni₅-Fe₁/CNT, and Ni₅-Fe₁/MgO.
